(19) United States
(12) Patent Application Publication
(10) Pub. No.: US 2021/0283017 A1
(43) Pub. Date: Sep. 16, 2021

(54) ASSEMBLY, DEVICES AND METHOD FOR PREPARING AND DISTRIBUTING MEDICINES IN PILLBOXES

(71) Applicant: DISTRAIMED, Montastruc la Conseillere (FR)

(72) Inventors: Michel FOUBET, Saint-Jean (FR); Olivier FOUBET, Bressols (FR); Gregory FOUBET, Toulouse (FR)

(21) Appl. No.: 16/343,939

(22) PCT Filed: Oct. 27, 2017
(86) PCT No.: PCT/EP2017/077597

§ 371 Date: Apr. 23, 2019

(30) Foreign Application Priority Data
Oct. 28, 2016 (FR) 1660543

(51) Int. Cl. A61J 7/00; A61G 12/00; G16H 20/13; G05B 15/02; B65G 1/137

(52) U.S. Cl. CPC A61J 7/0084; A61G 12/001; G16H 20/13; A61J 2205/60; B65G 1/1371; A61J 2205/10; A61J 2205/30; G05B 15/02

(57) ABSTRACT

The invention relates to devices for preparing (10) and distributing (40) medicines to a plurality of patients (32), said devices respectively comprising at least one preparation holder (16), one distribution holder (43) and receptacles (15) in which pillboxes (12) are inserted. The preparation and distribution holders are adapted to receive identical receptacles and the computing means of each distribution device access a database shared with the computing means of a preparation device. The invention relates to an assembly comprising at least one preparation device and at least one distribution device, as well as to a method for preparing and distributing medicines which is implemented using these devices.

The invention relates to a device for preparing medicines in pillboxes for the purpose of distributing them to a plurality of patients such as patients residing in the same establishment, in particular a medico-social establishment such as a retirement home, a home for handicapped persons, a rehabilitation or healthcare establishment (e.g. thalassotherapy), a sanatorium, a hospital, a clinic . . . The invention also relates to a device for distributing these medicines, to an assembly of such devices for preparing and distributing medicines and to a method for preparing and distributing medicines to a plurality of patients.

For example, WO2012/062986 discloses a device for preparing medicines corresponding to a prescription for a patient in the form of pillboxes forming a one-piece package comprising a plurality of packaging cells, each packaging cell containing a group, named unitary medicine dose, of at least one medicine intended to be administered to a single patient at a given time of the day, named dose time. In this known device, a packing apparatus fills various cells of a pillbox with a unitary medicine dose for each cell. In a parallel manner, the different parameters of this packing operation are verified and recorded in a centralised database using a computer system. For example, for each pillbox, the database receives a unique identifier of the pillbox, the name of the patient for whom it is intended, the prescription used, the number and type of medicines in each cell, the traceability information of these medicines, etc. A plurality of pillboxes can thus correspond to the same patient depending on the duration of the prescription and the period to which the pillbox corresponds. Taking into account the means necessary for the preparation, the preparation of pillboxes is generally carried out in a pharmacy, e.g. in a centralised pharmacy serving a plurality of medico-social establishments. The pillboxes thus prepared are then dispatched to the establishments in which they will be used by the staff responsible for distribution to the patients.

At this stage, various errors prejudicial to the safety and efficiency of the distribution of the medicines may occur. For example, pillboxes intended for one establishment may be sent to another establishment, causing at least some disorganisation of the distribution in the two establishments. Moreover, since the treatment of a patient extends over a period sufficiently long for two identical pillboxes to be prepared for the same patient there is a risk that two different care-givers will each use a different pillbox for this patient. Furthermore, the preparation of the distribution of the medicines can prove to be complicated if the doses corresponding to a dose time are separated from the pillbox before distribution, e.g. in an infirmary serving one floor of an establishment . . .

In order to avoid these errors, if they occur, leading to prejudicial consequences, the cited document provides for the use of a distribution device comprising portable computer terminals connected to the centralised database by a wireless connection in order to validate the correct distribution of the unitary dose of medicines to the right patient and to proceed with timestamping the distribution.

However, difficulty remains when identifying, among the various prepared pillboxes or fractions of prepared pillboxes, which one corresponds to the patient to be served. This search takes time and can be a source of error or irritation for the staff. Another solution consists of preparing the pillboxes according to an order corresponding to the order of the distribution round, which can prove to be time-consuming for the care staff during preparation of the distribution and to be restrictive if, for some reason, the order of the round has to be changed.

US 2009/0152291 describes an automatic sorting and packaging device for medicines and does not relate either to the preparation of pillboxes or the use of pillboxes for the distribution of medicines to a plurality of patients.

The invention thus aims to provide devices for preparing and distributing medicines to a plurality of patients, which facilitate the task of those involved.

The invention also aims to improve the safety of operations for preparing, transporting and distributing medicines.

In order to do this the invention relates, according to one aspect, to a device for preparing medicines in pillboxes for a plurality of patients according to claim 10. Said shared database is accessible at least for writing by said first computing means, and thus forms part thereof.

By virtue of such a preparation device, the pillboxes prepared and identified by the packing apparatus are arranged directly inside a uniquely identified receptacle. Each pillbox corresponds to a predetermined patient, i.e. has at least one code for identification of a single patient. Each pillbox is itself identified and located within the receptacle by the first computing means, these means comprising e.g. a computer connected to a memory containing a database bringing together the information relating to the patients, to the prescriptions, to the medicines used (batches, expiry dates, etc.), to the care establishments for which the pillboxes are intended, etc. Consequently, suitable programming of the first computing means permits the possible generation of an alarm if an unidentified or unauthorised pillbox is inserted into a given receptacle. For example, an alarm can be generated if two pillboxes corresponding to the same patient (e.g. prepared for two distinct periods) are arranged in the same receptacle.

In certain possible advantageous embodiments, the preparation holder allows not only the identification and locating of a pillbox in the receptacle to be recorded but also the operator to be informed, according to e.g. an item of information relative to a patient, at his location within the care establishment, as to the receptacle and position in the receptacle where he is to insert the pillbox which has just been prepared in the packing apparatus. The preparation device in accordance with the invention thus makes it possible to organise the future distribution of the medicines in the most effective manner possible.

According to another aspect, the invention also relates to a device for distributing medicines to a plurality of patients according to claim 12. In particular, said shared database is accessible at least for reading by said second computing means and is shared with the computing means of a device for preparing medicines—in particular with said first computing means of a device for preparing medicines in accordance with the invention.

By virtue of a device for distributing medicines in accordance with the invention, the distribution of the medicines to a group of patients, e.g. the patients on one floor of a care establishment, is simplified, rendered safe and quicker. In fact, the caregiver merely needs to take the receptacle corresponding to the group of patients in question, to check the identification thereof and to place it in a distribution holder fitted to a mobile distribution unit. The distribution of the medicines no longer needs to follow a pre-established itinerary since, no matter which patient is identified by the identification means, e.g. by the reading of an optically read code (in particular bar codes or two-dimensional codes) on the door of the room of said patient or the selection of the patient from a list displayed on a screen, the distribution holder, in collaboration with the information relating to the receptacle, provided by the shared database, created when the receptacle is being filled and accessible at least for reading by said second computing means fitted to the mobile unit, immediately indicates the location of the corresponding pillbox to the caregiver. It is no longer necessary to search for this pillbox in the group of prepared pillboxes, and the risks of error are minimised.

The invention also relates to an assembly of devices in accordance with the invention for preparing and distributing medicines to a plurality of patients. It thus relates in particular to an assembly of devices according to claim 1.

The performance of an assembly of devices for preparing and distributing medicines in accordance with the invention is greatly improved since the preparation device and the distribution device use identical receptacles and holders adapted to receive such identical receptacles. Furthermore, the sharing of information using a common database between the preparation device used in a main pharmacy and one or more distribution devices distributed over one or more establishments receiving the patients makes it possible to minimise the risks of errors and to improve the traceability of the preparation and the distribution of medicines.

In accordance with certain embodiments of the invention, each pillbox comprising a plurality of packaging cells, each packaging cell containing an group, named unitary dose of medicines, of at least one medicine, each pillbox bears a code for identification of the pillbox, each preparation holder is adapted to be able to read and record each identification code of each pillbox contained in each receptacle received in this preparation holder, and each distribution holder is adapted to be able to read each identification code of each pillbox contained in each receptacle received in this distribution holder.

In certain embodiments of the invention, the pillboxes are of the single-use type (i.e. they can be thrown away after use), e.g. in the form of transparent blister packs. Furthermore, in certain embodiments of the invention, each pillbox comprises a plurality of packaging cells, and each packaging cell of a pillbox can be separated from the other packaging cells of the pillbox, preferably before the packaging cell is opened. For example, each packaging cell can be formed of a transparent blister pack dimple or well closed by a removable seal, the whole packaging cell being connected by a perforation line to another adjacent packaging cell of the pillbox. Thus each pillbox is formed of at least one—in particular only one—row of wells with removable covers, e.g. in the form of self-adhesive labels. Nevertheless, the invention applies to pillboxes formed in any other manner.

According to one embodiment of such an assembly in accordance with the invention, this assembly further comprises secure packaging means for a set of receptacles, these packaging means being adapted to permit transportation of such a set of receptacles between a preparation site and a distribution site and for storage at the distribution site. By virtue of these secure packaging means, e.g. in the form of multi-packs capable of receiving four receptacles, it is possible to envisage distributions of medicines, e.g. by dose time, in which the four receptacles each correspond to a distinct dose time for the same group of patients.

In certain advantageous embodiments of the invention, each receptacle comprises a plurality of defined locations adapted to receive only a single pillbox in each case. Each receptacle can thus be organised in the form of a box comprising a plurality of parallel runners on its side walls, each pair of runners being able to hold a single pillbox. The number of pairs of runners is adapted to the average accommodation capacity of care establishments, e.g. of the order of 25 patients per floor.

In certain advantageous embodiments of the invention, each holder comprises means for detecting the presence of a pillbox in at least some of the locations—in particular in each location—of a receptacle when this receptacle is placed in the holder. Thus, the filling state of the receptacle can be checked during preparation and, similarly, the integrity of the receptacle and of the pillboxes placed therein can be checked when the receptacle is placed in a distribution holder. It is thus possible to ensure that no pillbox has been lost during transportation between the preparation site and the distribution site.

In certain advantageous embodiments of the invention, each receptacle comprises unique identification means readable by the first and second computing means. Consequently, each receptacle can be reliably and unequivocally identified when it is in use.

In certain advantageous embodiments of the invention, the identification means comprise at least one code for reading in a contactless manner—in particular for optical or RFID reading. These identification means are preferably analogous to the identification means used to identify the patients when the medicines are being distributed.

Alternatively or additionally, in certain advantageous embodiments of the invention, the identification means comprise a memory integral with the receptacle and adapted to be read by the holder in which the receptacle is placed. Thus an electronic circuit comprising a memory, e.g. a USB stick, integral with the receptacle can be plugged into a suitable socket provided in the holder when the receptacle is inserted therein. The memory can comprise, in addition to a unique identification code of the receptacle, complementary information as to its content (e.g. a copy of the fraction of the database relating to the pillboxes contained in the receptacle . . . ).

In certain advantageous embodiments of the invention, each receptacle comprises means for locking the pillboxes in the receptacle when this receptacle is not placed in a holder. Such locking means make it possible to lock each pillbox in place in a receptacle at least when the receptacle is not placed in a holder. By virtue of the use of means for individual or collective locking of the pillboxes in the receptacle, which are active at least when this receptacle is not placed in a holder, it is possible to prevent the loss of a pillbox during transport handling between the preparation site and the distribution site. Such means for locking the pillboxes in a receptacle can equally be means of the type which are manually activated into the locking position, means of the type which are automatically activated into the locking position when the receptacle is not placed in a holder, or even means of the type semi-automatically activated into the locking position (i.e. by means of a manual action combined with an automatic operation). Such means for locking the pillboxes in a receptacle can equally be means of the type which are manually activated into the unlocking position (in which the pillboxes can be removed from the receptacle), means of the type which are automatically activated into the unlocking position when the receptacle is placed in a holder, or even means of the type semi-automatically activated into the unlocking position (i.e. by means of a manual action combined with an automatic operation).

In certain embodiments of the invention, a distribution device in accordance with the invention is characterised in that the mobile distribution unit comprises means for supplying electric power to the second computing means and the distribution holder. By fitting the mobile units (which are e.g. distribution trolleys) with an electric power supply (formed e.g. by one or a plurality of rechargeable batteries), it is possible to supply electric power to all of the means used for distribution, i.e. the distribution holder, the second computing means, the identification code readers, etc. It is thus also possible to have a plurality of mobile units, some of which are being charged while the others are being used for distribution.

In certain embodiments of the invention, said mobile distribution unit comprises means for reading a contactlessly read code, these means being associated with the second computing means and adapted to read codes for identification of a patient, codes for identification of a receptacle and identification codes on each pillbox. By virtue of the use of the same means for reading the different identification codes and/or information used in the distribution method in accordance with the invention, e.g. a reader for optically read codes (e.g. bar codes or two-dimensional codes) in the form of a laser scanning bar code reader, the process of distributing medicines can be accelerated, the caregiver not needing to change the reading tool to identify the patient and pillboxes and the distributed medicines sequentially. Of course, said reading means do not rule out the possibility of using, as a variant or in combination, other data input means such as a keyboard/mouse and screen assembly, or a touch screen, which are connected to the second computing means to permit the selection of a patient from a list, possibly supplemented by photographs.

In certain embodiments of the invention, said mobile distribution unit comprises a timestamping clock for the distribution of a unitary dose of medicines to a patient. It is thus possible to check and validate the correspondence at least between the distribution time and the dose time provided for each pillbox.

In certain embodiments of the invention, the second computing means of the mobile distribution unit comprise communication means adapted to communicate with a computer network in order to access said shared database at least for reading. Said second computing means are preferably adapted also to access said shared database for writing. By virtue of the use of wireless communication means, e.g. via a radio link such as wi-fi, Bluetooth®. . . , it is possible to access the shared database in real time and to record therein e.g. that the patient has actually taken the medicines, the date and time of distribution of the medicines, etc. As a variant or in combination and in particular with the intention of limiting exposure to electromagnetic waves, the second computing means can be connected to the computer network by cable.

In certain embodiments of the invention, the second computing means of the mobile distribution unit also comprise at least one memory in which a local copy of the shared database is recorded. In the case where the second computing means are connected to the network by cable or if a real time wireless connection between the mobile distribution unit for medicines and the database is impossible, e.g. owing to the configuration of the premises, the second computing means of the mobile distribution unit thus comprise a memory permitting a copy of the database to be loaded, the relevant distribution information (timestamping, actual taking of the medicine, etc.) to be written thereto, then, once distribution is complete, the shared database to be updated. Furthermore, such a copy of the shared database makes it possible to overcome a possible break in communication between the mobile distribution unit and the communication network permitting the connection with the shared database.

The invention also relates to a device for preparing medicines which is adapted to form part of an assembly in accordance with the invention. The invention also relates to a device for distributing medicines which is adapted to form part of an assembly in accordance with the invention.

The invention also relates to a method for preparing and distributing medicines to a plurality of patients, implemented by virtue of an assembly in accordance with the invention. It thus also relates to a method for preparing and distributing medicines to a plurality of patients according to claim 19.

In a method, assembly and preparation device in accordance with the invention, said first computing means are adapted to be able to access, at least for writing, said shared database in order to permit, during the preparation step, at least the recording of each item of information for identification and locating of each pillbox in each receptacle. In certain embodiments in accordance with the invention, said first computing means are also adapted to be able to access, also for reading, said shared database.

In a method, assembly and distribution device in accordance with the invention, said second computing means are adapted to be able to access, at least for reading, said shared database. In certain embodiments in accordance with the invention, said second computing means are adapted to be able to access, also for writing, said shared database.

The invention also relates to a device for preparing medicines, a device for distributing medicines, an assembly of devices for preparing and distributing medicines, and a method for preparing and distributing medicines which are characterised, in combination or not in combination, by all or some of the features mentioned above or below. However they are formally presented, unless explicitly stated otherwise, the different features mentioned above or below should not be considered to be closely or inextricably linked with each other, the invention being able to relate to only one of these features, or only part of one of these features, or even any group, combination or juxtaposition of all or some of these features.

Other aims, features and advantages of the invention will become apparent in view of the following description and the accompanying drawings, in which:

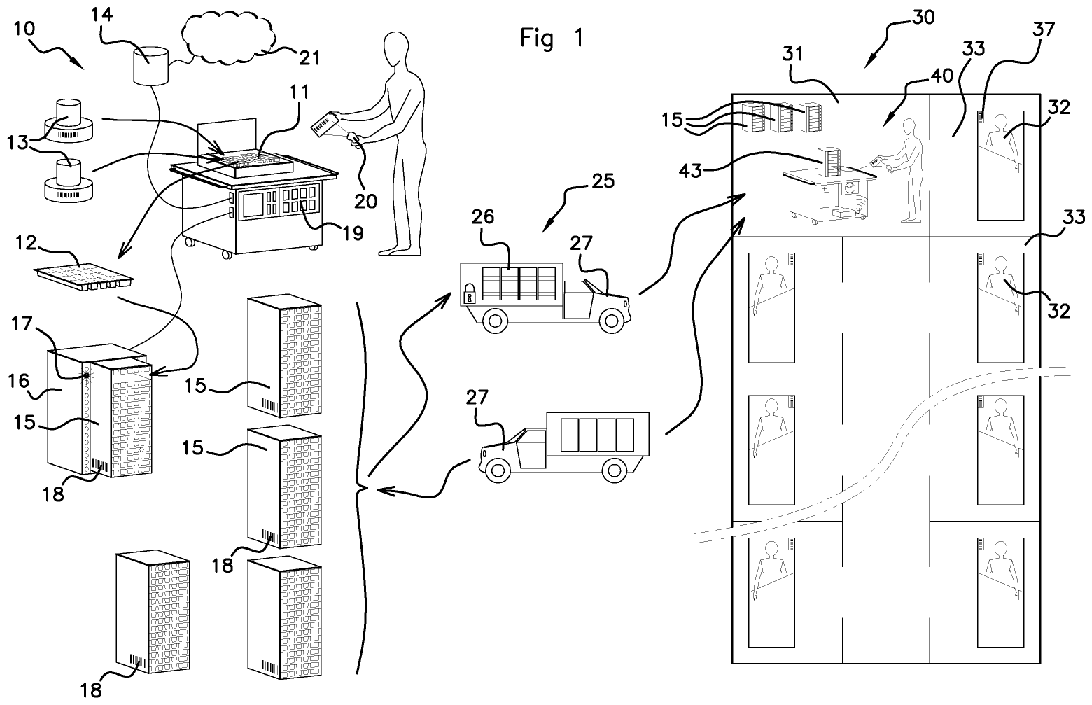

FIG. 1 illustrates an operating diagram of the devices for preparing, transporting and distributing medicines in accordance with the invention;

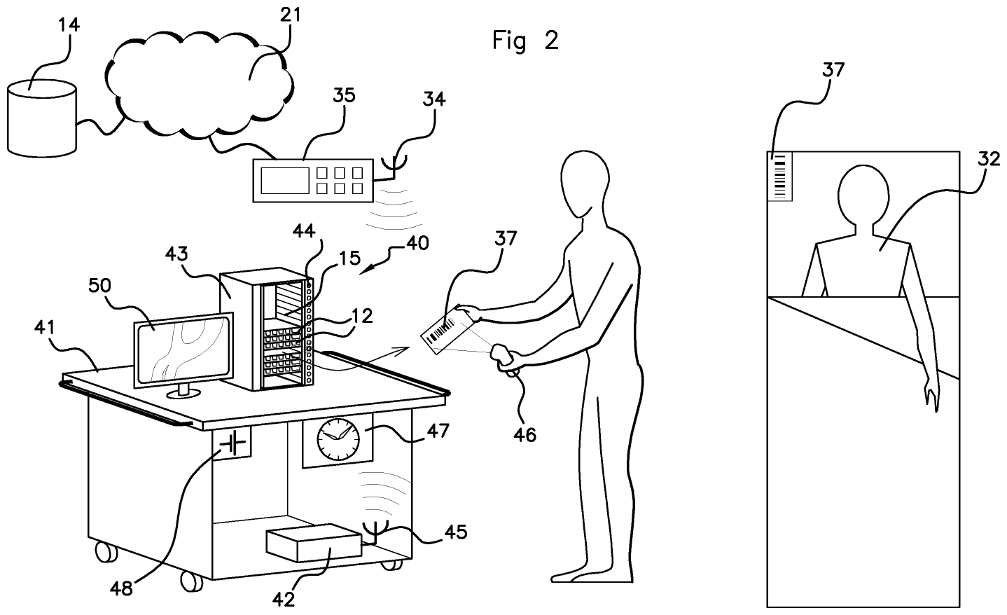

FIG. 2 schematically illustrates a device for distributing medicines in accordance with the invention;

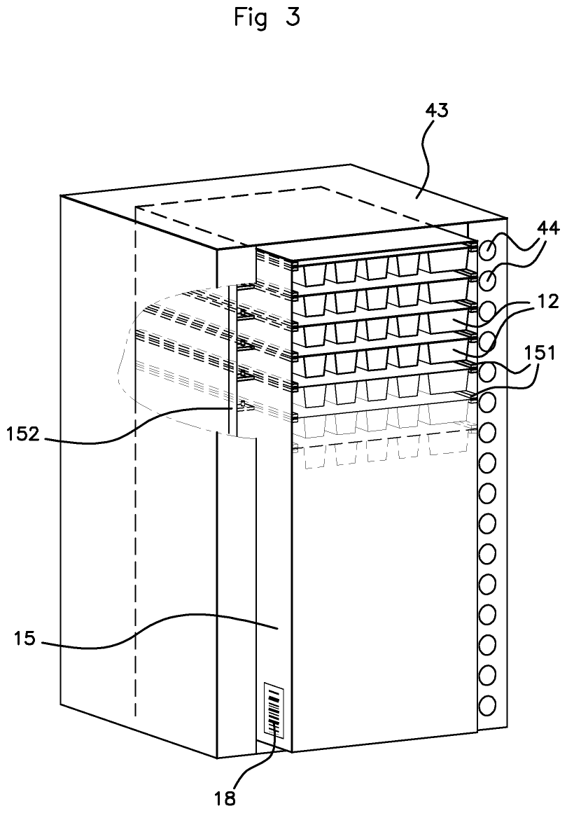

FIG. 3 illustrates a preparation or distribution holder associated with a receptacle in accordance with the invention;

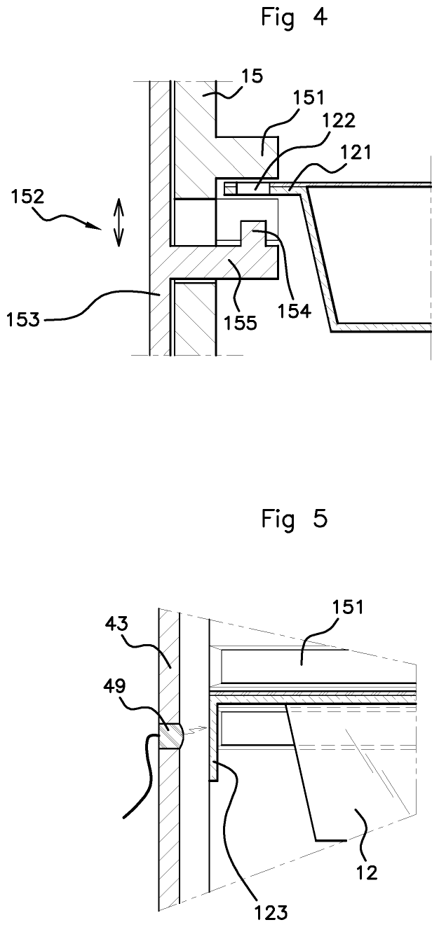

FIGS. 4 and 5 respectively illustrate embodiment details of a holder or of a receptacle in accordance with the invention.

In the example of FIG. 1, the left-hand side of the drawing shows a device for preparing medicines in pillboxes for the purpose of distributing them to patients, this device being generally designated by the reference sign 10, located within a preparation site, e.g. a pharmacy. At the centre of the drawing and generally designated by the reference sign 25, a transport device is shown permitting the medicines and the pillboxes prepared by the preparation device 10 to be safely dispatched in the direction of a distribution site 30 which is e.g. a care establishment in which patients reside.

The preparation site and the distribution site can be spaced apart from one another as illustrated, the pillboxes 12 prepared at the first site thus being transported, e.g. by vehicles 27, to the second site. However, there is nothing to prevent the two sites being located in the immediate proximity of one another or even constituting a single site, the pillboxes being prepared in the establishment housing the patients.

The pillboxes are advantageously single-use pillboxes (i.e. they can be thrown away after use) e.g. in the form of transparent blister packs having a plurality of packaging cells, each packaging cell containing a unitary dose of medicines for a patient. Each packaging cell of a pillbox can be formed of a transparent blister pack dimple or well closed by a removable seal formed by a printed self-adhesive cover, and may or may not be separable from the other packaging cells of the pillbox. Each packaging cell may or may not be compartmentalised in order to keep the different medicines of a single unitary dose of medicines separate from one another. Any other pillbox embodiment can be used within the invention.

The preparation device 10 comprises a packing apparatus 11 adapted to prepare pillboxes 12 from boxes of loose medicines 13, and to do so according to medical prescriptions specific to each patient. A packing apparatus 11 of this type is described, e.g. in WO2012/062986. The packing apparatus 11 is associated with first computing means comprising e.g. a computer 19 connected to a database 14. For each pillbox 12, the computer 19 records in the database 14 a code for identification of the pillbox, the identity of the patient for whom this pillbox is intended, the contents of the pillbox, the traceability information of the medicines used to fill the pillbox, etc.

Each pillbox is thus uniquely identified, e.g. by means of an RFID label or even by one or a plurality of optically read codes printed on at least one seal closing a dimple of the pillbox and/or on each seal closing each dimple of the pillbox.

The computer 19 is also connected to a preparation holder 16 adapted to receive at least one receptacle 15. In the illustrated example, each preparation holder 16 is adapted to receive a single receptacle 15, but there is nothing to prevent provision being made for a single preparation holder 16 to be able to receive a plurality of adjacent receptacles.

Each receptacle 15 is in the form of a generally parallelepiped box having an opening for insertion/extraction of a plurality of pillboxes 12 in/from this receptacle. Each receptacle 15 has a plurality of locations for receiving pillboxes, each reception location being adapted to receive just one pillbox.

Each preparation holder 16 is also in the form of a generally parallelepiped box having an opening for insertion/extraction of each receptacle 15 intended to be placed in this holder 16.

In the illustrated example, each preparation holder 16 comprises means for reading an identification code 18 of each receptacle 15 when the receptacle is being inserted into the holder, e.g. by means of an RFID label reader or optical reader or even an electronic circuit adapted to read a memory integral with the receptacle. The preparation holder 16 also comprises means, e.g. lights 17 permitting each cell of the receptacle 15 to be identified and indicated.

Thus, once a pillbox 12 has been filled by the packing apparatus 11, the computer 19 indicates to the operator where in the receptacle 15 the pillbox 12 must be arranged, e.g. by lighting the light 17 of the preparation holder 16 corresponding to the location provided for this pillbox in the receptacle 15. As will become clear hereinunder in relation to FIG. 5, the holder 16 can be provided with means for detecting the presence of a pillbox in each cell of the receptacle and can check the insertion of the pillbox in the provided and indicated location.

The information relating to the identification code 18 of the receptacle 15 and the information relating to the identification and locating of each pillbox 12 in the receptacle 15 during placement in the preparation holder 16 are transmitted by the holder to the computer 19 which retranscribes them in the database 14.

The preparation device 10 thus makes it possible to bring together the pillboxes 12 produced by the packing apparatus 11 in receptacles 15 and to identify in which receptacle and at which location in this receptacle each pillbox has been arranged. This information is associated with the information relating to each pillbox in the database 14.

The database 14 is shared e.g. with the care establishments which place orders using a remote link, e.g. via the Internet 21. Alternatively, when the receptacle 15 is provided with a memory integral with the receptacle, the fraction of the database 14 relating specifically to this receptacle and the pillboxes inserted therein can be transferred into the memory of the receptacle to be used subsequently, during the distribution of the medicines to the patients.

The preparation device 10 can comprise a plurality of holders 16 permitting numerous receptacles 15 to be prepared in parallel. It is thus possible to prepare a plurality of pillboxes for the same patient and to place each of them in a separate receptacle, e.g. when the pillboxes 12 are arranged to comprise only doses corresponding to a predetermined dose time (morning, noon, evening, bedtime . . . ), each pillbox can be placed in a receptacle corresponding to this predetermined dose time. The preparation device 10 can also be used to prepare receptacles 15 each corresponding, for one group of patients, to a day of the week, or to a week depending on the capacity of the pillboxes used.

The receptacles 15 thus prepared are then sorted by destination establishments and dispatched thereto in transport vehicles 27. In an advantageous manner, the receptacles 15 are brought together in a secure package 26, each package corresponding to a destination establishment and containing a plurality of receptacles, e.g. four receptacles for a given period (a day or a week), each receptacle corresponding to a dose time (morning, noon, evening, bedtime).

The receptacles 15 are delivered to the distribution site 30, e.g. in an infirmary 31 on one floor of the care establishment accommodating a plurality of patients 32, each housed in a room 33. The empty receptacles used during previous distributions and stored in the infirmary 31 are returned to the preparation site for reuse.

The infirmary 31 also holds a device 40 for distributing medicines. This distribution device 40, as will be seen in relation to FIG. 2, comprises a mobile distribution unit in the form of a trolley 41 on castors, able to be moved by the care staff from one room 33 to another. The trolley 41 is equipped with second computing means in the form of a computer 42. The computer 42 comprises wireless communication means 45 such as a WI-FI and/or Bluetooth® link . . . to a computer network of the care establishment comprising an antenna 34 and a router 35 connected to the Internet 21, thus permitting a direct connection via the Internet to a database 14 shared by the device 10 for preparing the medicines. Of course, with the intention of minimising the exposure of the patients and staff to electromagnetic waves, the computer 42 also comprises means for connection to the network and to the shared database 14 via a cable connection e.g. using an RJ45 or USB cable. The computer 42 can also comprise a memory capable of recording a copy of at least a fraction of the database 14 corresponding to the receptacle(s) 15 delivered by the preparation site to the distribution site so as to be able to operate even if there is a break in connection, either between the trolley 41 and the computer network 34 of the care establishment, or between the computer network 34 of the care establishment and the database 14 of the preparation device 10.

The device 40 for distributing medicines also comprises means for inputting data into the computer 42, e.g. a keyboard/mouse/screen assembly or even a touch screen 50, which are supplemented, as applicable, by means for reading a contactlessly read code, e.g. in the form of a reader 46 for optical codes (bar code, “Datamatrix” two-dimensional code) which is connected to the computer 42 by a wireless link in order to permit the care staff to move in the proximity of the trolley 41 without being hampered by a cable. The reader 46 is adapted to read a label 37 for identification of the patient 32, e.g. placed at the head of the bed of the patient or on the door of his room. The reader 46 transmits the identification information of the patient to the computer 42. The patient can also be identified by being selected from a list displayed on the touch screen 50, the list being able to be supplemented by photographs of the patient for positive identification of said patient.

The distribution device 40 also comprises a medicine distribution holder 43 fixed to the trolley 41 and connected to the computer 42. The distribution holder 43 is adapted to receive a receptacle 15 comprising pillboxes 12 arranged in the receptacle. The distribution holder 43 is also in the form of a generally parallelepiped box having an opening for insertion/extraction of a receptacle 15 previously prepared in a preparation holder 16 of the preparation device 10.

The trolley 41 can also comprise electric power supply means in the form of a rechargeable battery 48 permitting the supply of power to the computer 42 and the distribution holder 43 as well as any other electrical equipment which could be associated with the distribution device 40. For example, the trolley may also comprise a clock 47 enabling a date and time of distribution of the medicines to be provided. Of course, the clock 47 can form part of the second computing means and be included in the computer 42.

Reference is made to FIGS. 3 to 5 of the attached drawing to describe in detail a preparation holder 16 or a distribution holder 43 and a receptacle 15 as well as the interaction therebetween. In the illustrated examples, the reference numbers correspond to a distribution holder 43. Nevertheless, the distribution holder 43 has numerous points in common with the preparation holder 16.

FIG. 3 illustrates a distribution holder 43 in which a receptacle 15 is partly inserted. The receptacle 15 is in the form of an parallelepiped box which is open on one of its main faces. The internal side walls of the receptacle 15 comprise a plurality of runners 151 adapted to permit the side edges 121 (FIG. 4) of pillboxes 12 to engage therein. The receptacles 15 usually comprise twenty five pairs of runners so as to hold twenty five different pillboxes each corresponding to one of the patients of the distribution zone, generally a floor of a care establishment.

On one of its faces, the receptacle 15 comprises an identification label 18 permitting the receptacle engaged in the holder to be identified. Means for reading the identification label 18 can be either the code reader 46 for a distribution trolley 41 or the code reader 20 for a preparation device 10 or even means specific to the holders 16 or 43, the reading of the identification label being effected upon insertion of the receptacle 15 into the holder. The identification label 18 can also be replaced or, better, supplemented by an electronic circuit (not illustrated) integral with the receptacle 15 and comprising a memory. The electronic circuit comprises a connector enabling it to be connected to a corresponding connector of the holder when the receptacle is being plugged into the holder. Consequently, the computer 42 can have access, via the holder 43, to the information for identification of the receptacle 15 as well as to other information relating to the content of the receptacle.

The distribution holder 43 comprises a column of lights 44, each corresponding to a pillbox of the receptacle 15. The same is true of the preparation holder 16 which also comprises a column of lights 17, each corresponding to a pillbox of the receptacle 15. The lights 44 have the function of indicating to the caregiver carrying out the distribution of medicines which is the pillbox to be used according to the identification of the patient made using the identification label 37 associated with each patient and the code reader 46 of the distribution trolley 41 or the selection of the patient from a list displayed on the screen 50 Similarly, the lights 17 of the preparation holder 16 have the function of indicating to the person preparing the medicines where to insert the pillbox just prepared in the receptacle 15.

The receptacle 15 can also comprise locking means 152 adapted to keep the pillboxes 12 in place as long as the receptacle 15 is not placed in the distribution holder 43. By way of the example illustrated in FIG. 4, the locking means 152 are composed of a mobile rod 153 extending over the whole height of the receptacle 15 and comprising fractions of runners 155 integral with the mobile rod 153 comprising a stud 154 adapted to cooperate with an orifice 122 provided in one of the side edges 121 of the pillboxes 12. The mobile rod 153 moves between two positions, a first position, named locked position, in which the fraction of runner 155 is aligned with the runner 151 and the stud 154 passes into the orifice 122 in the side edge 121 of the pillbox 12 preventing same from being withdrawn from the receptacle 15, and a second position, named free position, in which the fraction of runner 155 is moved such that the stud 154 releases the pillbox 12. The mobile rod 153 is preferably held in the locked position by a spring (not illustrated) and moved in the direction of the free position upon insertion of the receptacle 15 into the distribution holder 43. By virtue of the locking means 152, pillboxes 12 inserted in a receptacle 15 cannot inadvertently be removed or fall from the receptacle 15 during handling and transportation operations.

The locking means 152, illustrated in the form of collective locking of the pillboxes 12 of a receptacle 15, can be in another form. In particular, the locking of the pillboxes can be envisaged individually, such as to release a pillbox only if it corresponds to the identification of the patient to whom the medicines are being distributed Similarly, the locking means can be borne by the pillboxes, e.g. in the form of an elastic tongue issuing from the side edge 121 of the pillbox 12 and cooperating by elastic anchoring in a hole in the runner 151. In this case, the distribution holder can comprise collective or individual unlocking means, e.g. in the form of a stud passing into the hole in the runner in order to eject the elastic tongue therefrom.

Moreover, the distribution holder 43 can comprise means for detecting the presence of a pillbox in its runner. By way of example, FIG. 5 illustrates a device for detecting the presence of a pillbox 12 in the form of a photoelectric sensor 49 placed e.g. on the rear face of the holder and cooperating with a vertical rim 123 integral with the pillbox 12. The reflection of the light signal emitted by the photoelectric sensor 49 on the vertical rim 123, likely to be metallised, thus permitting a signal indicating the presence of the pillbox 12 in this runner to be obtained.

Other variations of the presence detection device can be envisaged, e.g. the photoelectric sensor 49 can be replaced by a contactless code reader capable of reading an identification label of the pillbox borne by the vertical rim 123. Such a variation can be particularly useful within the scope of a preparation holder 16 to permit verification of the identification of the pillbox inserted in a runner indicated by a light 17 during filling of the receptacle 15.

The operation of the preparation device 10 and distribution device 40 in accordance with the invention in relation to a medicine preparation and distribution method in accordance with the invention using these devices is e.g. as follows.

In a first step of preparation, which generally takes place in a centralised pharmacy for a plurality of care establishments, pillboxes 12 are prepared by a packing apparatus 11 according to prescriptions issued by the doctors to the patients of an establishment. Information relating to the medicines thus prepared, to the identity of the patient for whom they are intended and the identification of the pillbox in which they have been prepared are recorded in a database 14 using a computer 19 of the preparation device 10.

In this preparation step, at least a first holder, named preparation holder 16, is used which is connected to the computer 19 and in which is placed a receptacle 15 adapted to receive a plurality of pillboxes 12. Since each receptacle comprises a unique identification label 18, this is read by a code reader 20 operated by a person preparing the medicines or even by reading means specific to the holder 16. The identification of the receptacle 15 during use is transmitted to the computer 19 and recorded in the database 14.

Once each pillbox 12 is prepared, it is inserted in the receptacle 15, e.g. a location determined by illumination of a corresponding light 17. The locations for each pillbox can be determined from information supplied by the care establishment as to the order of the distribution rounds. The locating of the location of each pillbox in the receptacle used is transmitted to the computer 19 and recorded in the database 14 in relation to the identification of the corresponding pillbox.

The database 14 thus contains, for each patient, the identification of the pillbox which is intended for him, its location in an identified receptacle, the information relating to the medicines contained in the pillbox etc.

The receptacles 15 intended for a distribution site 30, e.g. a predetermined care establishment, are brought together in a secure package 26 and transported to this establishment.

During a distribution step, a distribution device 40 is used, comprising a mobile trolley 41 on which is fixed a second holder, named distribution holder 43, connected to a computer 42 which is itself connected to the database 14 by wireless communication means and/or via the Internet or containing a full or partial copy of this database.

A receptacle 15 is then placed in the distribution holder 43 and the data input means, e.g. the code reader 46 connected to the computer 42, are used to read the identification borne by the label 18 of the receptacle. The computer 42 can thus be connected to the database 14 and record all the data of said database relating to the receptacle 15 used for the distribution. Alternatively or additionally, these data can be contained in a memory integral with the receptacle 15.

The caregiver then moves with the mobile trolley 41 to each patient. For each patient the identity of the patient is determined by selection from a list of patients displayed on the screen 50 or is read using the code reader 46 and an identification label 37 placed in the proximity of the patient, e.g. on the door of his room or at the head of his bed. On the basis of this identification of the patient, the computer 42 accesses, in the database 14, the location in the receptacle 15 of the pillbox 12 corresponding to the patient identified.

According to this location, the holder 43 indicates the location of the pillbox in the receptacle 15 by lighting the corresponding light 44.

The caregiver can thus directly access the pillbox corresponding to the patient and provide said patient with the medicines corresponding to the dose in question. At the same time, a number of other checks can be made, e.g. by rereading information written as code on the closure seal of the well of the pillbox 12, thus making it possible to do a double check that the medicines/patient and present time/planned dose time correspond. Similarly, information relating to distribution of the medicines to the patient as well as timestamping of this distribution can be transmitted to the computer 42 which retranscribes them into the database 14.

It is thus possible to obtain full traceability of the preparation and of the distribution of medicines to the patient, including which medicines have been prepared and administered, at what time, etc. Of course, this description is given merely by way of illustrative example, and a person skilled in the art will be able to made numerous modifications thereto apart from the variants mentioned in the description above, without departing from the scope of the invention, such as e.g. placing memories in each receptacle 15, these memories comprising not only the identification of the receptacle but also the part of the data of the database corresponding to this receptacle so as to avoid having a link between the database 14 and the distribution device 40.

The invention can cover numerous variants and applications other than those described above. In particular, it goes without saying that, unless stated otherwise, the different features of the embodiments described above do not have to be considered as being combined and/or closely and/or inextricably linked with each other, but in contrast considered as simple juxtapositions. Furthermore, the features of the different embodiments described above can form, in their entirety or in part, any different juxtaposition or any different combination.

1/ Assembly of devices for preparing and distributing medicines to a plurality of patients, said assembly comprising:
at least one device for preparing medicines in pillboxes for said plurality of patients, comprising:
at least one packing apparatus adapted to fill at least one cell of a one-piece package, named pillbox, this cell being specific to a patient of said plurality of patients, with a group, named unitary dose of medicines, of at least one medicine intended to be administered to this patient at a given time during the day, named dose time, each pillbox having a plurality of packaging cells and corresponding to a specific patient,
computing means, named first computing means, adapted to record information relating to said medicines, to the pillbox and to the patient for whom they are intended,
at least one receptacle adapted to receive a plurality of pillboxes which can be inserted in this receptacle once they have been prepared, each pillbox inserted in a receptacle corresponding to a specific patient,
at least one holder, named preparation holder, connected to said first computing means and adapted to receive at least one of said receptacles which is placed in said preparation holder, said preparation holder being adapted to record an identification element of each receptacle placed in said preparation holder as well as identification and locating information for each pillbox in each receptacle in a shared database,
at least one device for distributing medicines to said plurality of patients, comprising:
a mobile distribution unit comprising computing means, named second computing means,
means for identification of a patient to be served, which are connected to said second computing means,
at least one receptacle comprising a unique identification element, each receptacle comprising a plurality of pillboxes, each pillbox corresponding to a specific patient,
at least one holder, named distribution holder, fitted to said mobile distribution unit and connected to said second computing means and adapted to receive one of said receptacles which is placed in said distribution holder, said distribution holder being adapted to identify the receptacle placed in said distribution holder and indicate a position of a pillbox in the receptacle corresponding to the identification of a patient to be served, from a shared database,
in that the preparation holder and distribution holder are adapted to receive identical receptacles, each distribution holder being adapted to be able to receive a receptacle previously prepared in a preparation holder,
and in that the first and second computing means share a common shared database comprising identification and content information relating to said receptacles.

2/ Assembly according to claim 1, wherein each pillbox comprises a plurality of packaging cells, each packaging cell containing a group, named unitary dose of medicines, of at least one medicine, each pillbox bears a code for identification of the pillbox, in that each preparation holder is adapted to be able to read and record each identification code of each pillbox contained in each receptacle received in this preparation holder, and in that each distribution holder is adapted to be able to read each identification code of each pillbox contained in each receptacle received in this distribution holder.

3/ Assembly according to any one of claim 1, it wherein said assembly further comprises secure packaging means for a set of receptacles, which is adapted to transport receptacles between a preparation site and a distribution site and for storage at the distribution site.

4/ Assembly according to claim 1, wherein each receptacle comprises a plurality of defined locations adapted to receive only a single pillbox in each case.

5/ Distribution assembly according to claim 1, characterised in that wherein each preparation holder and each distribution holder comprises means for detecting the presence of a pillbox in each location of a receptacle when this receptacle is installed in the holder.

6/ Assembly according to claim 1, wherein each receptacle comprises unique identification means readable by reading means associated with the first and second computing means.

7/ Assembly according to claim 6, wherein the identification means comprise at least one contactlessly read code.

8/ Assembly according to of claim 6, wherein said identification means comprise a memory integral with the receptacle and adapted to be able to be read by the holder in which the receptacle is placed.

9/ Assembly according to claim 1, wherein each receptacle comprises means for locking the pillboxes in the receptacle when this receptacle is not placed in a holder.

10/ Device for preparing medicines in pillboxes for a plurality of patients, comprising:
at least one packing apparatus adapted to fill at least one cell of a one-piece package, named pillbox, this cell being specific to a patient of said plurality of patients, with a group, named unitary dose of medicines, of at least one medicine intended to be administered to this patient at a given time during the day, named dose time, each pillbox having a plurality of packaging cells and corresponding to a specific patient,
computing means, named first computing means, adapted to record information relating to said medicines, to the pillbox and to the patient for whom they are intended,
wherein said device comprises:
at least one receptacle adapted to receive a plurality of pillboxes which can be inserted in this receptacle once they have been prepared, each pillbox inserted in a receptacle corresponding to a specific patient,
at least one holder, named preparation holder, connected to said first computing means and adapted to receive at least one of said receptacles which is placed in said preparation holder, said preparation holder being adapted to record an identification element of each receptacle placed in said preparation holder as well as identification and locating information for each pillbox in each receptacle in a shared database.

11/ Device for preparing medicines according to claim 10, wherein said device is adapted to form part of an assembly according to one of claim 1.

12/ Device for distributing medicines to a plurality of patients, comprising:
a mobile distribution unit comprising computing means, named second computing means,
means for identification of a patient to be served, which are connected to said second computing means,
wherein said device comprises:
at least one receptacle comprising a unique identification element comprising a plurality of packages, named pillboxes, each pillbox being a one-piece packaging having a plurality of packaging cells and corresponding to a specific patient,
at least one holder, named distribution holder, fitted to said mobile distribution unit, connected to said second computing means and adapted to receive one of said receptacles which is placed in said distribution holder, said distribution holder being adapted to identify the receptacle placed in said distribution holder and to indicate a position of a pillbox in the receptacle corresponding to the identification of a patient to be served, from a shared database.

13/ Distribution device according to claim 12, wherein the mobile distribution unit comprises means for supplying electric power to the second computing means and the distribution holder.

14/ Distribution device according to claims 12, wherein the mobile distribution unit comprises means for reading a contactlessly read code, these reading means being associated with the second computing means and adapted to read codes for identification of a patient, codes for identification of a receptacle and information codes borne by each pillbox.

15/ Distribution device according to claim 12, wherein the mobile distribution unit comprises a timestamping clock for the distribution of a unitary dose of medicines to a patient.

16/ Distribution device according to claim 12, wherein the second computing means of the mobile distribution unit comprise communication means adapted to communicate with a computer network in order to access said shared database.

17/ Distribution device according to claim 12, wherein the second computing means of the mobile distribution unit also comprise at least one memory in which a local copy of said shared database is recorded.

18/ Distribution device according to claim 12, wherein said distribution device is adapted to form part of an assembly according to claim 1.

19/ Method for preparing and distributing medicines to a plurality of patients, in which, for each patient of said plurality of patients, medicines intended for this patient being packaged in at least one one-piece package, named pillbox, comprising a plurality of packaging cells, each packaging cell containing a group, named unitary dose of medicines, of at least one medicine intended to be administered to this patient at a given time of the day, named dose time, each pillbox having a plurality of packaging cells and corresponding to a specific patient,
wherein:
during a preparation step,
at least one holder, named preparation holder, is used, which is connected to computing means, named first computing means, adapted to be able to access a shared database,
at least one receptacle adapted to receive a plurality of pillboxes inserted in this receptacle once they have been prepared is placed in said holder, and an identification element of each receptacle placed in said preparation holder is recorded in said database,
at least one pillbox is inserted in each receptacle and identification and locating information for each pillbox in each receptacle is recorded in said shared database,
during a distribution step,
a holder, named distribution holder, is used, which is connected to second computing means adapted to share said shared database,
a receptacle previously prepared in a preparation holder is placed in the distribution holder,
data input means are used to read identification information of the receptacle,
for each patient, the data input means are used to obtain an identification element of the patient,
depending on the identification of the patient, the second computing means consult said shared database and control the distribution holder to indicate the location in the receptacle of the pillbox corresponding to the patient.
